(19) United States
(12) Patent Application Publication
(10) Pub. No.: US 2022/0298158 A1
(43) Pub. Date: Sep. 22, 2022

(54) POLYMORPH OF VENETOCLAX AND METHOD FOR PREPARING THE POLYMORPH

(71) Applicant: Johnson Matthey Public Limited Company, London (GB)

(72) Inventors: Amanda BUIST, Cambridge (GB); Alex EBERLIN, Cambridge (GB)

(21) Appl. No.: 17/634,335

(22) PCT Filed: Aug. 12, 2020
(86) PCT No.: PCT/GB2020/051913

§ 371 Date: Feb. 10, 2022

(30) Foreign Application Priority Data
Aug. 14, 2019 (GB) 1911627.6

(51) Int. Cl. C07D 471/04; A61P 35/00; A61P 35/02

(52) U.S. Cl. CPC C07D 471/04; A61P 35/00; A61P 35/02; C07B 2200/13

(57) ABSTRACT

The present invention relates to a polymorph of venetoclax, to a process for its preparation, and to pharmaceutical compositions containing the polymorph.

The present invention relates to a polymorph of venetoclax, to a process for its preparation, and to pharmaceutical compositions containing the polymorph.

### BACKGROUND

Venetoclax has the chemical name of 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino] phenyl}sulfonyl)-2-(1H-pyrrolo[2,3-b]pyridine-5-yloxy)benzamide and the chemical structure illustrated below:

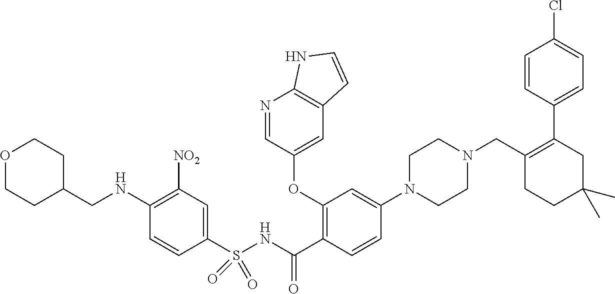 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Venetoclax is sold under the brand name Venclexta in the US and in the EU as Venclyxto. Venetoclax is an antineoplastic agent and Venclyxto monotherapy is indicated for treating chronic lymphocytic leukaemia (CLL) when other treatments have failed or are unsuitable. As the number of patients with CLL is low, the disease is considered rare and Venclyxto has been designated an orphan medicine in the EU.

U.S. Pat. No. 8,722,657 (to Abbvie Inc.) describes salts and crystalline forms of 4-(4-{[2-(4-chlorophenyl)-4,4- dimethylcyclohex-1-en-1-yl]methyl}piperazin-1-yl)-N-({3-nitro-4-[(tetrahydro-2H-pyran-4-ylmethyl) amino]phenyl}sulfonyI)-2-(1 H-pyrrolo[2,3-b]pyridine-5-yloxy)benzamide. U.S. Pat. No. 8,722,657 does not describe the venetoclax anhydrate disclosed herein or a process for its preparation.

Information about the solid-state properties of a drug substance is important. For example, different forms may have differing solubilities. Also, the handling and stability of a drug substance may depend on the solid form.

Polymorphism may be defined as the ability of a compound to crystallise in more than one distinct crystal species and different crystal arrangements of the same chemical composition are termed polymorphs. Polymorphs of the same compound arise due to differences in the internal arrangement of atoms and have different free energies and therefore different physical properties such as solubility, chemical stability, melting point, density, flow properties, hygroscopicity, bioavailability, and so forth. The compound venetoclax may exist in a number of polymorphic forms and many of these forms may be undesirable for producing pharmaceutically acceptable compositions. This may be for a variety of reasons including lack of stability, high hygroscopicity, low aqueous solubility and difficulty in handing.

### DEFINITIONS

The term “about” or “approximately” means an acceptable error for a particular value as determined by a person of ordinary skill in the art, which depends in part on how the value is measured or determined. In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4%, 3%, 2%, 1%, or 0.5% of a given value or range. In certain embodiments and with reference to X-ray powder diffraction two-theta peaks, the terms “about” or “approximately” means within ±0.2° 2θ.

The term “ambient temperature” means one or more room temperatures between about 15° C. to about 30° C., such as about 15° C. to about 25° C.

The term “anti-solvent” refers to a first solvent which is added to a second solvent to reduce the solubility of a compound in that second solvent. The solubility may be reduced sufficiently such that precipitation of the compound from the first and second solvent combination occurs.

The term “crystalline” and related terms used herein, when used to describe a compound, substance, modification, material, component or product, unless otherwise specified, means that the compound, substance, modification, material, component or product is substantially crystalline as determined by X-ray diffraction. See, e.g., Remington: The Science and Practice of Pharmacy, 21st edition, Lippincott, Williams and Wilkins, Baltimore, Md. (2005); The United States Pharmacopeia, 23rd ed., 1843-1844 (1995).

The terms “polymorph,” “polymorphic form” or related term herein, refer to a crystal form of one or more molecules of venetoclax, or venetoclax molecular complex thereof that can exist in two or more forms, as a result different arrangements or conformations of the molecule(s) in the crystal lattice of the polymorph.

The term “pharmaceutical composition” is intended to encompass a pharmaceutically effective amount of venetoclax of the invention and a pharmaceutically acceptable excipient. As used herein, the term “pharmaceutical compositions” includes pharmaceutical compositions such as tablets, pills, powders, liquids, suspensions, emulsions, granules, capsules, suppositories, or injection preparations.

The term “excipient” refers to a pharmaceutically acceptable organic or inorganic carrier substance. Excipients may be natural or synthetic substances formulated alongside the active ingredient of a medication, included for the purpose of bulking-up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance, such as by facilitating powder flowability or non-stick properties, in addition to aiding in vitro stability such as prevention of denaturation over the expected shelf life.

The term “patient” refers to an animal, preferably a patient, most preferably a human, who has been the object of treatment, observation or experiment. Preferably, the patient has experienced and/or exhibited at least one symptom of the disease or disorder to be treated and/or prevented. Further, a patient may not have exhibited any symptoms of the disorder, disease or condition to be treated and/prevented, but has been deemed by a physician, clinician or other medical professional to be at risk for developing said disorder, disease or condition.

The terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more therapeutic agents to a patient with such a disease or disorder. In some embodiments, the terms refer to the administration of a molecular complex provided herein, with or without other additional active agents, after the onset of symptoms of a disease.

The term “overnight” refers to the period of time between the end of one working day to the subsequent working day in which a time frame of about 12 to about 18 hours has elapsed between the end of one procedural step and the instigation of the following step in a procedure.

### BRIEF DESCRIPTION OF THE FIGURES

Certain aspects of the embodiments described herein may be more clearly understood by reference to the drawings, which are intended to illustrate but not limit, the invention, and wherein:

FIG. 1 is a representative XRPD pattern of venetoclax anhydrate.

FIG. 2 is a representative TGA thermogram and a DSC thermogram of venetoclax anhydrate.

FIG. 3 a representative ¹H-NMR spectrum of venetoclax anhydrate.

FIG. 4 is a representative GVS isotherm plot of venetoclax anhydrate. The solid black triangle symbol ( [symbol] ) represents the cycle 1 sorption isotherm plot. The black cross symbol ( [symbol] ) represents the cycle 1 desorption isotherm plot. The grey cross within a black square symbol ( [symbol] ) represents the cycle 2 sorption isotherm plot. The solid back diamond symbol ( [symbol] ) represents the cycle 2 desorption isotherm plot. The solid black square symbol ( [symbol] ) represents the cycle 3 sorption isotherm plot.

FIG. 5 is a representative XRPD pattern overlay of venetoclax anhydrate before and after the GVS experiment.

FIG. 6 is representative XRPD overlay of venetoclax anhydrate before storage (bottom), venetoclax anhydrate after storage at 25° C./97% RH (relative humidity) for 7 days (middle), and venetoclax anhydrate after storage at 40° C./75% RH for 7 days (top).

FIG. 7 is a representative polarized light microscopy (PLM) image of venetoclax anhydrate.

### DESCRIPTION OF THE INVENTION

The present invention seeks to overcome the disadvantages associates with the prior art. It has been discovered that venetoclax can be prepared in a well-defined and consistently reproducible anhydrous crystalline form. Moreover, a reliable and scalable method for producing this anhydrous crystalline form has been developed. The venetoclax polymorph provided by the present invention is useful as an active ingredient in pharmaceutical formulations. In certain embodiments, the anhydrous crystalline form is purifiable. In certain embodiments and depending on time, temperature and humidity, the anhydrous crystalline form is stable. In certain embodiments, the anhydrous crystalline form is easy to isolate and handle. In certain embodiments, the process for preparing the anhydrous crystalline form is scalable.

The crystalline form described herein may be characterised using a number of methods known to the skilled person in the art, including single crystal X-ray diffraction, X-ray powder diffraction (XRPD), differential scanning calorimetry (DSC), thermal gravimetric analysis (TGA), infrared spectroscopy, Raman spectroscopy, nuclear magnetic resonance (NMR) spectroscopy (including solution and solid-state NMR). The chemical purity may be determined by standard analytical methods, such as thin layer chromatography (TLC), gas chromatography, high performance liquid chromatography (HPLC), and mass spectrometry (MS).

In one aspect, the present invention provides a crystalline form of venetoclax, which is crystalline venetoclax anhydrate.

Venetoclax is a free base and, as the form of the present invention is a crystalline anhydrate, it is not a salt, hydrate or solvate.

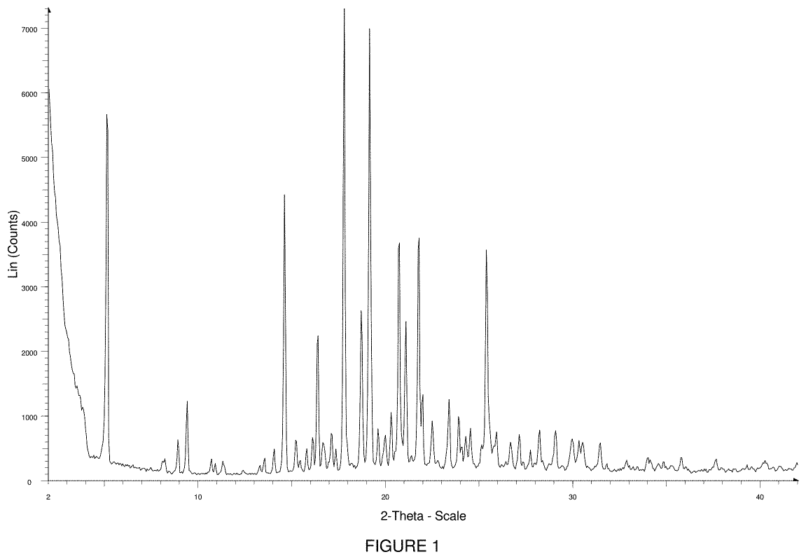

The anhydrate may have an X-ray powder diffraction pattern comprising one or more peaks (for example 1, 2, 3, 4, 5, 6, 7, or 8 peaks) selected from the group consisting of about 5.1, 8.1, 8.9, 9.4, 10.7, 11.0, 11.3, 13.3, 13.6, 14.0, 14.6, 15.2, 15.5, 15.8, 16.1, 16.4, 16.7, 17.1, 17.4, 17.8, 18.7, 19.2, 19.6, 20.0, 20.3, 20.8, 21.1, 21.8, 22.1, 22.5, 22.9, 23.4, 23.9, 24.3, 24.5, 25.4, 25.9, 26.7, 27.2, 27.7, 28.2, 29.1, 30.0, 30.4, and 30.6 degrees two-theta ±0.2 degrees two-theta. In one embodiment, the anhydrate may have an X-ray powder diffraction pattern comprising peaks at about 5.1, 8.9, 9.4, 14.6, and 17.8 degrees two-theta ±0.2 degrees two-theta. In one embodiment, the anhydrate may have the X-ray powder diffraction pattern substantially as shown in FIG. 1.

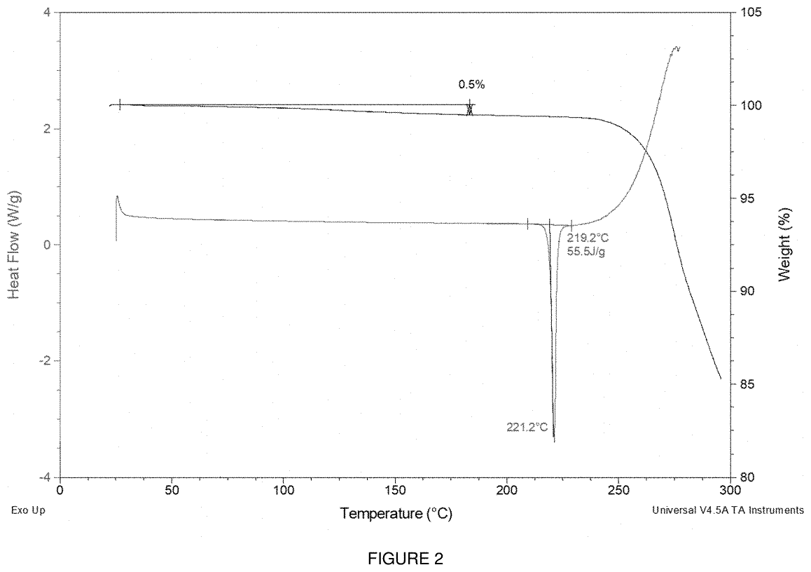

The anhydrate may have a DSC thermogram comprising an endothermic event with an onset at about 219.2° C. In one embodiment, the anhydrate may have a DSC thermogram substantially as shown in FIG. 2.

The anhydrate may have a TGA thermogram comprising about 0.5% mass loss when heated from about ambient temperature to about 175° C. In one embodiment, the anhydrate may have a TGA thermogram substantially as shown in FIG. 2.

Crystalline venetoclax anhydrate may be prepared by a process comprising the steps of:
(a) contacting venetoclax with a solvent which is heptane, optionally in combination with isopropyl acetate;
(b) forming a solution or suspension of venetoclax in the solvent; and
(c) recovering venetoclax anhydrate as a crystalline solid.

In one embodiment, the venetoclax which is contacted with a solvent is venetoclax hydrate.

In one embodiment, the solvent is heptane. In another embodiment, the solvent is a combination of heptane and isopropyl acetate. In one embodiment, the v/v ratio of heptane: isopropyl acetate is about 1 ml: about 1 ml.

In certain embodiment, the ventoclax anhydrate of the invention is pure. In certain embodiments, the chemical purity of the anhydrate is ≥90%, ≥91%, ≥92%, ≥93%, ≥94%, ≥95% or higher. In certain embodiments, the chemical purity of the anhydrate is 95%. In certain embodiments, the chemical purity of the anhydrate is ≥96%. In certain embodiments, the chemical purity of the anhydrate is 97%. In certain embodiments, the chemical purity of the anhydrate is ≥98%.

The quantity of solvent is not particularly limiting provided there is enough solvent to dissolve the venetoclax and form a solution, or suspend the venetoclax. The w/v ratio of venetoclax to solvent may be in the range of about 1 g of venetoclax : about 30 to about 150 ml solvent, such as about 1 g of venetoclax: about 40 to about 145 ml solvent, for example, about 1 g of venetoclax: about 45 to about 140 ml solvent.

The venetoclax may be contacted with the solvent at ambient temperature or less. Alternatively, the venetoclax may be contacted with the solvent at a temperature greater than ambient i.e. greater than 30° C. and below the boiling point of the reaction mixture. The boiling point of the reaction mixture may vary depending on the pressure under which the contacting step is conducted. In one embodiment, the contacting step is carried out at atmospheric pressure (i.e. 1.0135×10⁵ Pa). In one embodiment, the contacting step may be carried out at one or more temperatures in the range of ≥ about 60° C. to ≤ about 85° C. In some embodiments, the contacting step is carried out at one or more temperatures ≥ about 60° C. In some embodiments, the contacting step is carried out at one or more temperatures ≥ about 61° C. In some embodiments, the contacting step is carried out at one or more temperatures ≥ about 62° C. In some embodiments, the contacting step is carried out at one or more temperatures ≥ about 63° C. In some embodiments, the contacting step is carried out at one or more temperatures ≥ about 64° C. In some embodiments, the contacting step is carried out at one or more temperatures ≥ about 65° C. In some embodiments, the contacting step is carried out at one or more temperatures ≥ about 66° C. In some embodiments, the contacting step is carried out at one or more temperatures ≥ about 67° C. In some embodiments, the contacting step is carried out at one or more temperatures ≥ about 68° C. In some embodiments, the contacting step is carried out at one or more temperatures ≤ about 85° C. In some embodiments, the contacting step is carried out at one or more temperatures ≤ about 84° C. In some embodiments, the contacting step is carried out at one or more temperatures ≤ about 83° C. In some embodiments, the contacting step is carried out at one or more temperatures ≤ about 82° C. In some embodiments, the contacting step is carried out at one or more temperatures ≤ about 81° C. In some embodiments, the contacting step is carried out at one or more temperatures ≤ about 80° C. In one embodiment, the contacting step is carried out at one or more temperatures in the range of ≥ about 68° C. to ≤ about 80° C. In one embodiment, the contacting step is carried out at a temperature of about 75° C. In one embodiment, the contacting step is carried out at a temperature of about 80° C. In one embodiment, the contacting step is carried out at one or more temperatures in the range of ≥ about 75° C. to ≤ about 80° C. for a period of time (e.g. about three days), and then at ambient temperature (e.g. room temperature) for a period of time (e.g. overnight).

The dissolution or suspension of venetoclax may be encouraged through the use of an aid such as stirring, shaking and/or sonication. Additional solvent may be added to aid the dissolution or suspension of the venetoclax.

The solution or suspension may then be cooled such that the resulting solution or suspension has a temperature below that of the solution or suspension step (b). The rate of cooling may be from about 0.05° C./minute to about 2° C./minute, such as about 0.5° C./minute to about 1.5° C./minute, for example about 1° C./minute. When a solution of venetoclax is cooled, a suspension may eventually be observed. When a suspension of venetoclax is cooled, no perceptible change in the appearance of the suspension may occur.

The solution or suspension may be cooled to ambient temperature or a temperature of less than ambient temperature. In one embodiment, the solution or suspension may be cooled to one or more temperatures in the range of ≥ about 0° C. to about ≤20° C. In some embodiments, the solution or suspension is cooled to one or more temperatures ≥ about 1° C. In some embodiments, the solution or suspension is cooled to one or more temperatures ≥ about 2° C. In some embodiments, the solution or suspension is cooled to one or more temperatures ≥ about 3° C. In some embodiments, the solution or suspension is cooled to one or more temperatures ≥ about 4° C. In some embodiments, the solution or suspension is cooled to one or more temperatures ≥ about 5° C. In some embodiments, the solution or suspension is cooled to one or more temperatures ≤ about 15° C. In some embodiments, the solution or suspension is cooled to one or more temperatures ≤ about 14° C. In some embodiments, the solution or suspension is cooled to one or more temperatures ≤ about 13° C. In some embodiments, the solution or suspension is cooled to one or more temperatures ≤ about 12° C. In some embodiments, the solution or suspension may be cooled to one or more temperatures ≤ about 11° C. In some embodiments, the solution or suspension is cooled to one or more temperatures ≤ about 10° C. In one embodiment, the solution or suspension is cooled to one or more temperatures in the range of about 5° C. to about 10° C.

In step (c), the venetoclax anhydrate is recovered as a crystalline solid. The crystalline anhydrate may be recovered by directly by filtering, decanting or centrifuging. If desired, the suspension may be mobilised with additional portions of the solvent prior to recovery of the crystalline solid.

Alternatively, a proportion of the solvent may be evaporated prior to recovery of the crystalline solid.

Howsoever the crystalline anhydrate is recovered, the separated anhydrate may be washed with solvent (e.g. heptane, isopropyl acetate, or a mixture thereof) and dried. Drying may be performed using known methods, for example, at temperatures in the range of about 10° C. to about 60° C., such as about 20° C. to about 40° C., for example, ambient temperature under vacuum (for example about 1 mbar to about 30 mbar) for about 1 hour to about 24 hours. It is preferred that the drying conditions are maintained below the point at which the anhydrate degrades and so when the anhydrate is known to degrade within the temperature or pressure ranges given above, the drying conditions should be maintained below the degradation temperature or vacuum.

Steps (a) to (c) may be carried out one or more times (e.g. 1, 2, 3, 4 or 5 times). When steps (a) to (c) are carried out more than once (e.g. 2, 3, 4 or 5 times), step (a) may be optionally seeded with crystalline venetoclax anhydrate which was previously prepared and isolated by the first iteration of steps (a) to (c).

Alternatively or in addition, when steps (a) to (c) are carried out more than once (e.g. 2, 3, 4 or 5 times), the solution or suspension formed in step (b) may be optionally seeded with crystalline venetoclax anhydrate (which was previously prepared and isolated by a method described herein).

In certain embodiment, the ventoclax anhydrate prepared by the process of the invention is pure. In certain embodiments, the chemical purity of the anhydrate is ≥90%, ≥91%, ≥92%, ≥93%, ≥94%, ≥95% or higher. In certain embodiments, the chemical purity of the anhydrate is ≥95%. In certain embodiments, the chemical purity of the anhydrate is ≥96%. In certain embodiments, the chemical purity of the anhydrate is ≥97%. In certain embodiments, the chemical purity of the anhydrate is ≥98%.

In another aspect, the present invention relates to a pharmaceutical composition comprising crystalline venetoclax anhydrate as described herein and a pharmaceutically acceptable excipient.

In another aspect, the present invention relates to a method for treating cancer in a patient comprising administering a therapeutically effective amount of crystalline venetoclax anhydrate as described herein to the patient. The method of treatment includes the treatment of chronic lymphocytic leukaemia.

In another aspect, the present invention relates to crystalline venetoclax anhydrate as described herein for use in treating cancer, such as the treatment of chronic lymphocytic leukaemia.

Embodiments and/or optional features of the invention have been described above. Any aspect of the invention may be combined with any other aspect of the invention, unless the context demands otherwise. Any of the embodiments or optional features of any aspect may be combined, singly or in combination, with any aspect of the invention, unless the context demands otherwise.

The invention will now be described further by reference to the following examples, which are intended to illustrate but not limit, the scope of the invention.

### EXAMPLES

### General

X-Ray Powder Diffraction (XRPD)

X-Ray Powder Diffraction patterns were collected on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consists of a single Göbel multilayer mirror coupled with a pinhole collimator of 0.3 mm.

The beam divergence, i.e. the effective size of the X-ray beam on the sample, was approximately 4 mm. A θ-θ continuous scan mode was employed with a sample—detector distance of 20 cm which gives an effective 2θ range of 3.2° — 29.7° . Typically the sample would be exposed to the X-ray beam for 120 seconds. The software used for data collection was GADDS for XP/2000 4.1.43 and the data were analysed and presented using Diffrac Plus EVA v15.0.0.0.

Proton Nuclear Magnetic Resonance (¹H-NMR)

NMR spectra were collected on a Bruker 400MHz instrument equipped with an auto-sampler and controlled by a DRX400 console. Automated experiments were acquired using ICON-NMR v4.0.7 running with Topspin v1.3 using the standard Bruker loaded experiments. For non-routine spectroscopy, data were acquired through the use of Topspin alone.

Samples were prepared in DMSO-d₆, unless otherwise stated. Off-line analysis was carried out using ACD Spectrus Processor 2014.

Differential Scanning Calorimetry (DSC)

DSC data were collected on a TA Instruments Q2000 equipped with a 50 position auto-sampler. The calibration for thermal capacity was carried out using sapphire and the calibration for energy and temperature was carried out using certified indium. Typically 0.5-3 mg of each sample, in a pin-holed aluminium pan, was heated at 10° C./min from 25° C. to 300° C. A purge of dry nitrogen at 50 ml/min was maintained over the sample.

The instrument control software was Advantage for Q Series v2.8.0.394 and Thermal Advantage v5.5.3 and the data were analysed using Universal Analysis v4.5A.

Thermo-Gravimetric Analysis (TGA)

TGA data were collected on a TA Instruments Q500 TGA, equipped with a 16 position auto-sampler. The instrument was temperature calibrated using certified Alumel and Nickel. Typically 5 — 10 mg of each sample was loaded onto a pre-tared aluminium DSC pan and heated at 10 ° C/min from ambient temperature to 350 ° C. A nitrogen purge at 60 ml/min was maintained over the sample.

The instrument control software was Advantage for Q Series v2.5.0.256 and Thermal Advantage v5.5.3 and the data were analysed using Universal Analysis v4.5A.

Polarised Light Microscopy (PLM)

Leica LM/DM polarised light microscope

Samples were studied on a Leica LM/DM polarised light microscope with a digital video camera for image capture. A small amount of each sample was placed on a glass slide, mounted in immersion oil and covered with a glass slip, the individual particles being separated as well as possible. The sample was viewed with appropriate magnification and partially polarised light, coupled to a λ false-colour filter.

Gravimetric Vapour Sorption (GVS)

Hygroscopicity of a solid material may be determined by means of gravimetric vapour sorption (GVS) analysis, sometimes known by dynamic vapour sorption (DVS) analysis. The experiment subjects a sample material which is held in a fine wire basket on a microbalance within a temperature and humidity controlled environment (chamber). Using the software, the collected data can then be processed to determine the isotherm points at the increment ranges specified during the experiment and show the overall water uptake of the material.

Sorption isotherms were obtained using a SMS DVS Intrinsic moisture sorption analyser, controlled by DVS Intrinsic Control software v1.0.1.2 (or v 1.0.1.3). The sample temperature was maintained at 25° C. by the instrument controls. The humidity was controlled by mixing streams of dry and wet nitrogen, with a total flow rate of 200 ml/min The relative humidity was measured by a calibrated Rotronic probe (dynamic range of 1.0−100%RH), located near the sample. The weight change, (mass relaxation) of the sample as a function of %RH was constantly monitored by the microbalance (accuracy ±0.005 mg).

Typically 5−20 mg of sample was placed in a tared mesh stainless steel basket under ambient conditions. The sample was loaded and unloaded at 40% RH and 25° C. (typical room conditions). A moisture sorption isotherm was performed as outlined below (2 scans giving 1 complete cycle). The standard isotherm was performed at 25° C. at 10% RH intervals over a 0−90% RH range. Data analysis was carried out using Microsoft Excel using DVS Analysis Suite v6.2 (or 6.1 or 6.0).

Method for SMS DVS Intrinsic experiments:

| Parameter | Value |
|---|---|
| Adsorption-Scan 1 | 40-90 |
| Desorption/Adsorption-Scan 2 | 90-0, 0-40 |
| Intervals (% RH) | 10 |
| Number of Scans | 4 |
| Flow rate (ml/min) | 200 |
| Temperature (° C.) | 25 |
| Stability (° C./min) | 0.2 |
| Sorption Time (hours) | 6 hour time out |

The sample was recovered after completion of the isotherm and re-analysed by XRPD.

Chemical Purity Determination by HPLC Purity analysis was performed on an Agilent HP1100 series system equipped with a diode array detector and using ChemStation software vB.04.03 using the method detailed below:

HPLC method for chemical purity determinations:

| Parameter | Value | | |
|---|---|---|---|
| Type of method | Reverse phase with gradient elution | | |
| Sample Preparation | 0.5 mg/ml in acetonitrile:water 1:1 and 2 μl of TFA | | |
| Column | Supelco Ascentis Express C18, 100 × 4.6 mm, 2.7 μm | | |
| Column Temperature (° C.) | 25 | | |
| Injection (μl) | 5 | | |
| Wavelength, Bandwidth (nm) | 255, 90 | | |
| Flow Rate (ml/min) | 2 | | |
| Phase A | 0.1% TFA in water | | |
| Phase B | 0.085% TFA in acetonitrile | | |
| Timetable | Time (min) | % Phase A | % Phase B |
| | 0 | 95 | 5 |
| | 6 | 5 | 95 |
| | 6.2 | 95 | 5 |
| | 8 | 95 | 5 |

### EXAMPLE 1

Venetoclax (96.6% pure) used as the starting material. It was characterised as being hydrate C as described in U.S. Pat. No. 8,722,657.

Venetoclax (96.6% pure, ca. 520 mg) was suspended in 25 ml (50 vol.) of heptane and stirred at 75° C. After stirring at 75° C. for 9 days, 20 ml of heptane was added to aid stirring. The bulk sample was stirred at 75° C. for a total of 14 days. The solid was filtered and dried under vacuum.

### EXAMPLE 2

Venetoclax (96.6% pure, ca. 514 mg) and seeds of material obtained as Example 1 were suspended in 35 ml (70 vol.) of heptane and stirred at 75° C. After ca. 30 minutes, additional seeds were added. The sample was stirred at 75° C. for ca. 1 hour then the temperature was increased to 80° C. The sample was stirred at 80° C. for 3 days then overnight at RT. The bulk sample was filtered and dried under suction for 15 minutes.

### EXAMPLE 3

Material as prepared in Example 2 (ca. 25 mg) was suspended in 1.75 ml (70 vol.) of heptane: isopropyl acetate (1:1) and stirred at 75° C. for 2 days. The solid was filtered and dried under suction for ca. 15 minutes prior to XRPD analysis.

### EXAMPLE 4

Venetoclax (96.6% pure, ca. 510 mg) was suspended in 70 ml (70 vol) of heptane: isopropyl acetate (1:1) at ambient conditions. The suspension was then heated to 80° C. Seeds of anhydrous venetoclax as prepared in Example 3, were added at 68° C. The sample was then cooled to 25° C. at 1° C. per minute. The sample was held at 25° C. for ca. 75 minutes then filtered under suction for 1.5 hours to give anhydrous venetoclax. The chemical purity of the anhydrous venetoclax was 98.4% as determined by HPLC.

Characterisation of venetoclax anhydrate:

FIG. 1 is a representative XRPD pattern of crystalline venetoclax anhydrate. The following table provides an XRPD peak listing for the crystalline venetoclax anhydrate of the invention:

| Angle | Intensity |
|---|---|
| 2-Theta ° | % |
| 5.1 | 77.4 |
| 8.1 | 4.4 |
| 8.9 | 8.4 |
| 9.4 | 16.7 |
| 10.7 | 4.4 |
| 11.0 | 3.4 |
| 11.3 | 3.9 |
| 13.3 | 3 |
| 13.6 | 4.6 |
| 14.0 | 6.5 |
| 14.6 | 60.5 |
| 15.2 | 8.4 |
| 15.5 | 4.1 |
| 15.8 | 6.5 |
| 16.1 | 8.9 |
| 16.4 | 30.5 |
| 16.7 | 7.9 |
| 17.1 | 9.8 |
| 17.4 | 6.5 |
| 17.8 | 100 |
| 18.7 | 35.9 |
| 19.2 | 95.7 |
| 19.6 | 10.8 |
| 20.0 | 9.4 |
| 20.3 | 14.3 |
| 20.8 | 50.3 |
| 21.1 | 33.6 |
| 21.8 | 51.3 |
| 22.1 | 18 |
| 22.5 | 12.5 |
| 22.9 | 4.1 |
| 23.4 | 17 |
| 23.9 | 13.4 |
| 24.3 | 9.2 |
| 24.5 | 10.9 |
| 25.4 | 48.7 |
| 25.9 | 10.2 |
| 26.7 | 8 |
| 27.2 | 9.6 |
| 27.7 | 6.3 |
| 28.2 | 10.5 |
| 29.1 | 10.4 |
| 30.0 | 8.6 |
| 30.4 | 8.3 |
| 30.6 | 7.9 |

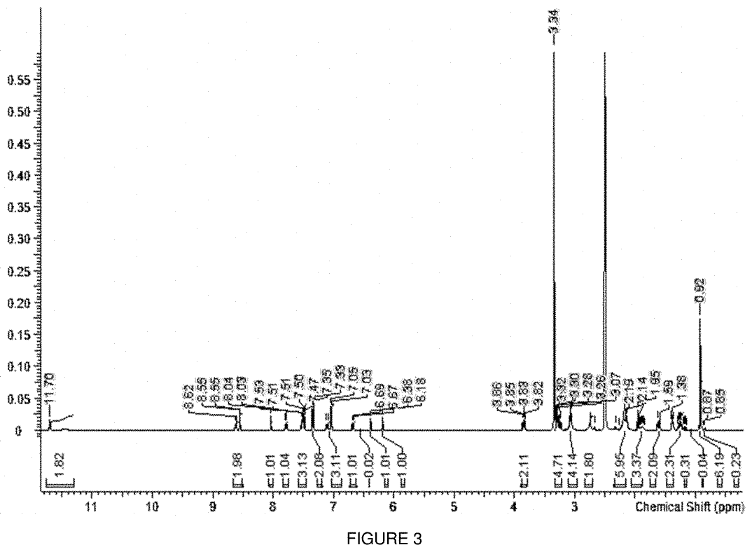

Crystalline venetoclax anhydrate was also characterised as follows:
TGA and DSC analysis (see FIG. 2);
¹H-NMR analysis (see FIG. 3);

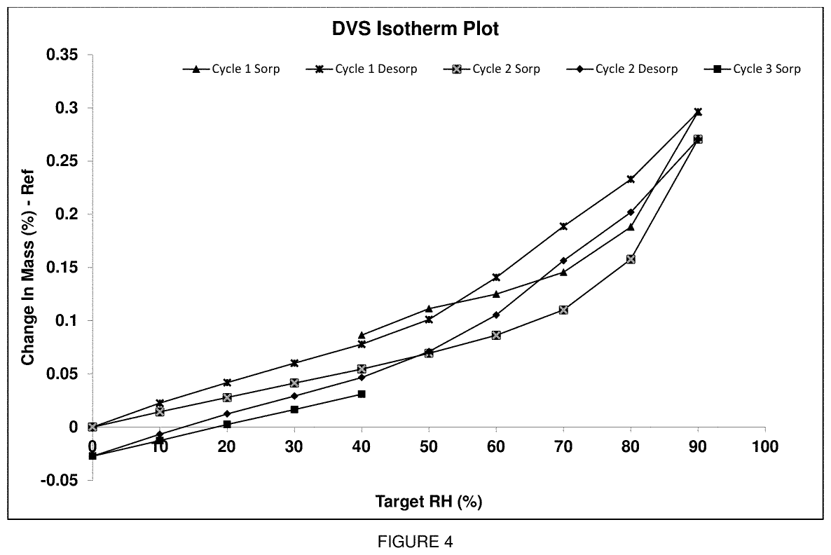

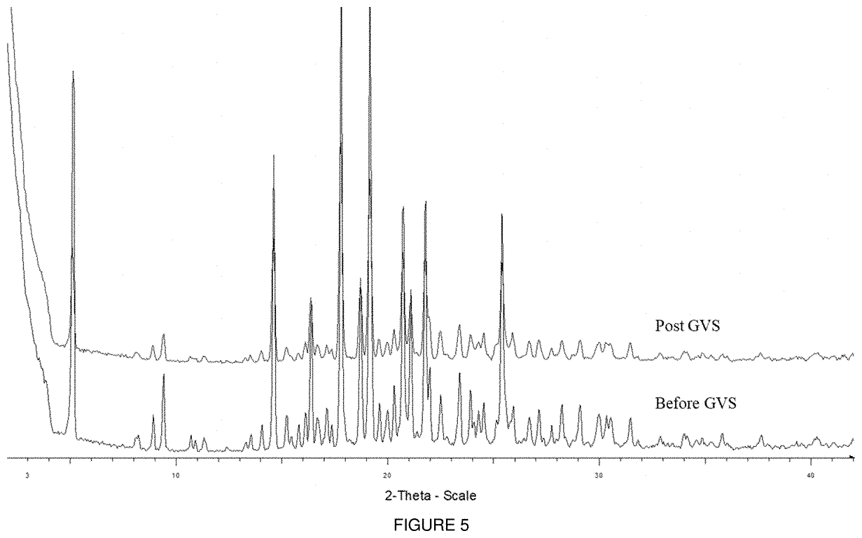

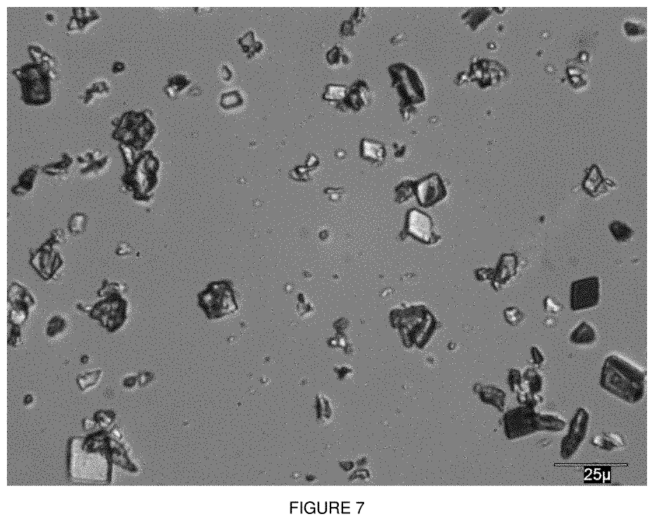

GVS isotherm analysis (see FIG. 4);
XRPD analysis before and after the GVS experiment (see FIG. 5); and
Polarized light microscopy (see FIG. 7).

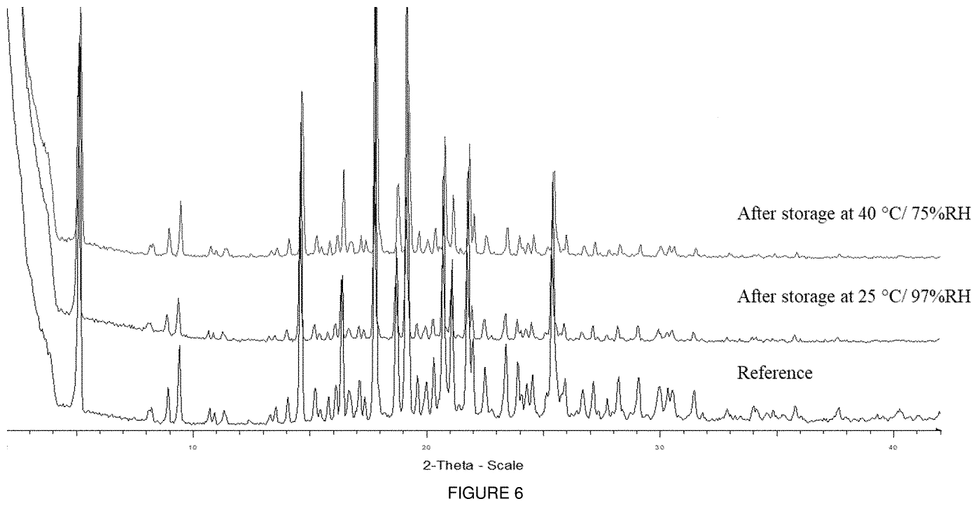

Stability studies of venetoclax anhydrate under two storage conditions were also carried out. FIG. 6 is representative XRPD overlay of venetoclax anhydrate before storage (bottom), venetoclax anhydrate after storage at 25° C/97% RH (relative humidity) for 7 days (middle), and venetoclax anhydrate after storage at 40° C/75% RH for 7 days (top). The anhydrate remains stable under two different temperature and humidity conditions for at least 7 days.

1. A crystalline form of venetoclax, which is crystalline venetoclax anhydrate.

2. A crystalline form of venetoclax according to claim 1 having an X-ray powder diffraction pattern comprising one or more peaks selected from the group consisting of about 5.1, 8.1, 8.9, 9.4, 10.7, 11.0, 11.3, 13.3, 13.6, 14.0, 14.6, 15.2, 15.5, 15.8, 16.1, 16.4, 16.7, 17.1, 17.4, 17.8, 18.7, 19.2, 19.6, 20.0, 20.3, 20.8, 21.1, 21.8, 22.1, 22.5, 22.9, 23.4, 23.9, 24.3, 24.5, 25.4, 25.9, 26.7, 27.2, 27.7, 28.2, 29.1, 30.0, 30.4, and 30.6 degrees two-theta ±0.2 degrees two-theta.

3. A crystalline form of ventoclax according to claim 2 having an X-ray powder diffraction pattern comprising peaks at about 5.1, 8.9, 9.4, 14.6, and 17.8 degrees two-theta ±0.2 degrees two- theta.

4. A crystalline form of ventoclax according to claim 3, which has an X-ray powder diffraction pattern substantially as shown in FIG. 1.

5. A crystalline form of venetoclax according to any one of claims 1 to 4, which has a DSC thermogram comprising an endothermic event with an onset at about 219.2° C.

6. A crystalline form of venetoclax according to claim 5, which has a DSC thermogram substantially as shown in FIG. 2.

7. A crystalline form of venetoclax according to any one of the preceding claims, which has a TGA thermogram comprising about 0.5% mass loss when heated from about ambient temperature to about 175° C.

8. A crystalline form of venetoclax according to claim 7, which has a TGA thermogram substantially as shown in FIG. 2.

9. A crystalline form of venetoclax according to any one of the preceding claims, which has a chemical purity of 98%.

10. A process for preparing crystalline venetoclax anhydrate, the process comprising the steps of:
(a) contacting venetoclax with a solvent which is heptane, optionally in combination with isopropyl acetate;
(b) forming a solution or suspension of venetoclax in the solvent; and
(c) recovering venetoclax anhydrate as a crystalline solid.

11. A pharmaceutical composition comprising crystalline venetoclax anhydrate as claimed in any one of claims 1 to 9 and a pharmaceutically acceptable excipient.

12. A method for treating cancer in a patient comprising administering a therapeutically effective amount of crystalline venetoclax anhydrate as claimed in any one of claims 1 to 9 to the patient.

13. A method for treatment according to claim 12, wherein the method of treatment is the treatment of chronic lymphocytic leukaemia.

14. Crystalline venetoclax anhydrate as claimed in any one of claims 1 to 9 for use in treating cancer.

15. Crystalline venetoclax according to claim 14 for use in the treatment of chronic lymphocytic leukaemia.
